(19) United States
(12) Patent Application Publication
(10) Pub. No.: US 2002/0024019 A1
(43) Pub. Date: Feb. 28, 2002

(54) Mask defect checking method and device for electron beam exposure

(76) Inventor: Ryoichi Matsuoka, Chiba-shi (JP)

Correspondence Address: ADAMS & WILKS, 31st Floor, 50 BROADWAY, NEW YORK, NY, 10004, US

(21) Appl. No.: 09/933,785

(22) Filed: Aug. 20, 2001

(30) Foreign Application Priority Data
Aug. 25, 2000 (JP) 2000-254970

(51) Int. Cl. G21G 5/00; A61N 5/00

(52) U.S. Cl. 250/492.3

(57) ABSTRACT

In order to check for defects in an electron beam exposure mask M, a mask signal S3 is acquired based on transmission electrons 2Ba acquired by two dimensional scanning of the electron beam exposure mask M by an electron beam scanning device 2, and a CAD signal S4 corresponding to a CAD graphic is acquired, synchronized with output of the mask signal S3 based on CAD data DT for making the electron beam exposure mask M. Defects in the electron beam exposure mask M are checked for defects based on comparison results of the mask signal S3 and the CAD signal S4

### BACKGROUND OF THE INVENTION

1. Field of the Invention

The present invention relates to a mask defect checking method and device for electron beam exposure.

2. Description of the Prior Art

In patterning processes for making various types of semiconductor, generally a mask forming a mask pattern on a transparent glass substrate is adopted, and used to perform patterning of photo-resist coated on a wafer with light rays in a region from visible light to ultra-violet as a light source. However, there have been advances in making circuit patterns ultra fine in recent years, and there has been a need for higher resolution in order to form circuit patterns in the order of nanometers, and this had led to exposure devices using an electron beam (EB) to be adopted instead of the above described light source.

As an exposure mask in the case of using an electron beam, an electron beam exposure mask such as a stencil mask formed by punching out the required exposure pattern from a silicon sheet, for example, is used.

In order to check for defects in the various exposure masks, such as those used in semiconductor manufacture, to determine if they are good or bad, in the related art a method of checking mask defects by comparing an optical image obtained using an electron microscope etc. or a mask image to be checked as an SEM image with a specified reference image, or a method using CAD data used in manufacturing the mask to check mask defects by comparing the mask image to be checked with a CAD mask image from CAD data, is adopted.

However, in the case of an electron beam exposure mask, if an ultra fine process is realized to form a mask pattern on a wafer of about 20 cm diameter and perform electron beam exposure, the number of these patterns will be enormous. As a result, if defect checking of the electron beam exposure mask is carried out using the related art method described above, as well as the fact that data acquisition of the mask images to be checked takes a lot of time, data transmission of the acquired mask image and image data comparison also require a lot of time. Accordingly, the overall checking time becomes extremely long, and in particular, there is a problem that this is not realistic in adopting production processes required to start up short-term mass production.

### SUMMARY OF THE INVENTION

An object of the present invention is to provide an electron beam exposure mask defect checking method and device that can be expected to increase the speed of defect checking of the electron beam exposure mask in order to solve the above described problems of the related art.

In order to solve the above problems, with the present invention, a mask signal corresponding to a mask shape based on a mask transmission electron signal acquired by two dimensional scanning of an electron beam exposure mask to be checked using an electron beam, this mask signal is compared with a CAD signal corresponding to CAD graphics for making the mask, and defects in the mask are checked based on the comparison results.

According to the present invention, there is provided a mask defect checking device for electron beam exposure, for checking defects of a mask used in electron beam exposure, comprising an electron beam scanner for two dimensional scanning of a mask to be checked using an electron beam in response to a given scanning signal, mask signal output means for outputting a mask signal in response to a mask shape based on transmission electrons passing through the mask from scanning of the electron beam, CAD signal output means for outputting a CAD signal showing a required mask shape in synchronism with output of the mask signal based CAD data for making the mask, and comparison means for comparing the mask signal and the CAD signal, wherein defects of the mask are checked based on output from the comparison means. Synchronization of the mask signal and the CAD signal can also be based on the scanning signal.

It is also possible for the mask signal output means to comprise a transmission electron detector for detecting the transmission electrons, and a sensitivity regulator for comparing an output signal from the transmission electron detector with a reference signal of a given fixed level to acquire the mask signal. Further, it is possible for mismatch information of the mask signal and the CAD signal from the comparison means to be taken out as a defect signal, and also to store the extracted defect signal in memory.

According to the present invention, there is also provided an electron beam exposure mask checking method, comprising the steps of acquiring a mask signal corresponding to a mask shape based on the mask transmission electron signal acquired by two dimensional scanning of a mask to be checked using an electron beam, comparing the mask signal with a CAD signal corresponding to CAD graphics for making the mask, and checking for defects in the mask based on the comparison results.

In this case, it is possible to synchronize the mask signal and the CAD signal based on a scanning signal for two-dimensional scanning of the electron beam.

### BRIEF DESCRIPTION OF THE DRAWINGS

FIG. 1 is a schematic diagram showing one example of an embodiment of a mask defect checking device of the present invention.

FIG. 2A shows the level of X direction scanning signal S1X.

FIG. 2B shows the level of Y direction scanning signal S1Y.

FIG. 3A shows part of the mask shape of the electron beam exposure mask M.

FIG. 3B shows an output signal S2 acquired when this mask shape section is scanned in the X direction as shown by the dotted line P in the drawing using an electron beam.

FIG. 3C shows a mask signal S3 is which is acquired varying in level in a binary manner in response to the mask shape shown by FIG. 3A.

FIG. 3D shows a CAD graphic which is the mask shape estimated based on the CAD data DT.

FIG. 3E shows the waveform of the CAD signal S4.

FIG. 3F shows the detect signal S6.

FIG. 4 is a drawing showing one example of checking result data.

### DETAILED DESCRIPTION OF THE PREFERRED EMBODIMENTS

An example of an embodiment of the present invention will now be described in detail, with reference to the drawings.

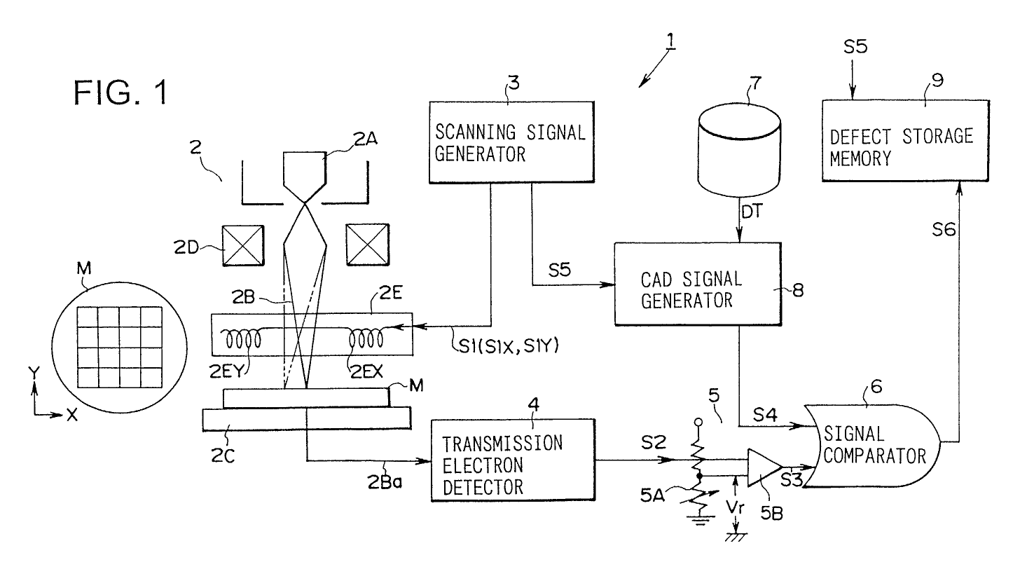

FIG. 1 is a schematic diagram showing one example of an embodiment of a mask defect checking device of the present invention. The mask defect checking device 1 is a device for checking defects in a mask pattern of an electron beam exposure mask M, and is provided with an electron beam scanning device 2 for two dimensional scanning of the electron beam exposure mask M using an electron beam. The electron beam scanning device 2 has a well known structure comprising an electron gun 2A, an electron lens 2D for focusing an electron beam 2B from the electron gun 2A on the electron beam exposure mask M mounted on a sample table 2C that is transparent to the electron beam, and a deflector 2E for two dimensionally scanning the electron beam 2B on the electron beam exposure mask M in X and Y directions, and a scanning signal S1 from a scanning signal generator 3 is provided to the deflector 2E.

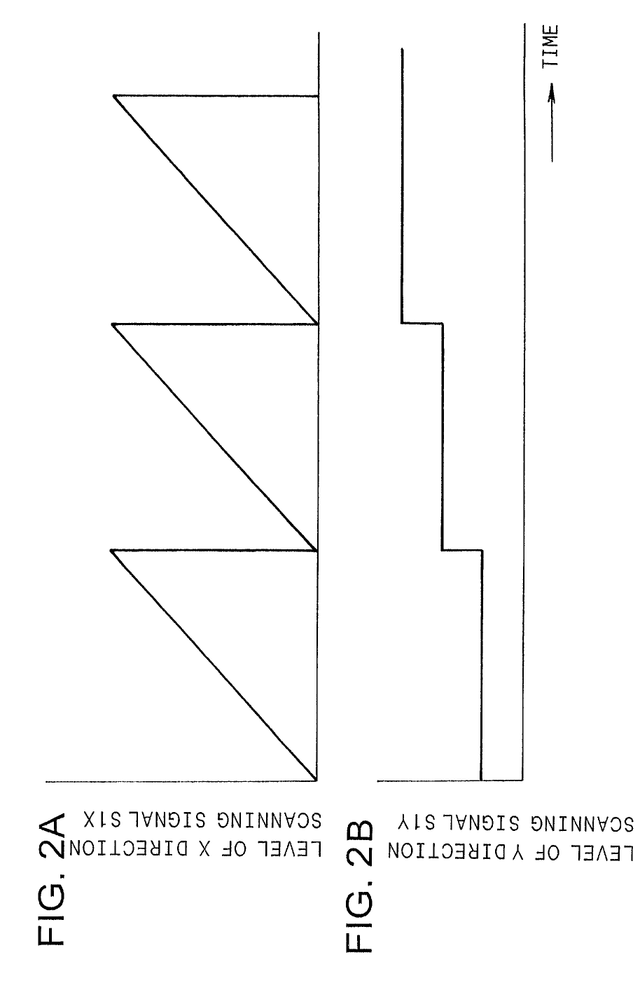

As shown in FIG. 2, the scanning signal S1 is made up of an X direction scanning signal S1X and a Y direction scanning signal S1Y, the X direction scanning signal S1X and the Y direction scanning signal S1Y are respectively applied to an X direction deflector coil 2EX and a Y direction deflector coil 2EY of the deflector 2E. The electron beam exposure mask M is therefore two dimensionally scanned in the X and Y directions using the electron beam 2B.

The electron beam exposure mask M, as exemplified in FIG. 1, has a well-known circular shape formed by punching out a necessary mask pattern on a thin silicon sheet. When the electron beam 2B is X-Y scanned in accordance with the scanning signal S1, transmission electrons 2Ba passing through the electron beam exposure mask M and reaching a lower surface 2Ca side of the sample table 2C are detected by the transmission electron detector 4. The transmission electrons 2Ba have information of the mask pattern of the electron beam exposure mask M, and a mask pattern of the electron beam exposure mask M, namely an output signal S2 appropriate to the mask shape, is output from the transmission electron detector 4, this output signal S2 being used for sensitivity regulation in the sensitivity regulator 5. With the embodiment shown in FIG. 1, the sensitivity regulator 5 performs voltage comparison of the level of the output signal S2 with a reference voltage Vr acquired by a variable resistive potential divider circuit 5A using the voltage comparator 5B, and this comparison output is output as a mask signal S3.

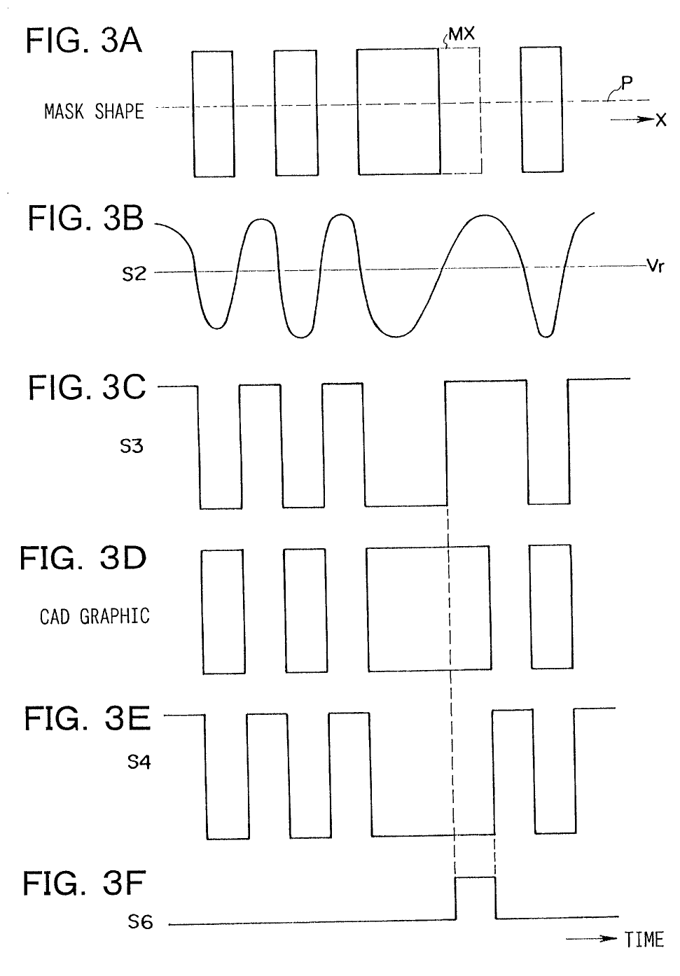

Operation of the sensitivity regulator 5 will now be described with reference to FIG. 3. FIG. 3A represents part of the mask shape of the electron beam exposure mask M, and an output signal S2 acquired when this mask shape section is scanned in the X direction as shown by the dotted line P in the drawing using an electron beam is represented by FIG. 3B. The output signal S2 is a signal from transmission electrons acquired by scanning the mask shape of FIG. 3A, and so the level of the signal S2 varies according to the mask shape. The output signal S2 is subjected to level comparison, by the voltage comparator 5B, with a reference voltage Vr having a level appropriately set by the variable resistive potential divider circuit 5A. In this way, the output signal is subjected to waveform shaping, and as shown by FIG. 3C a mask signal S3 is acquired varying in level in a binary manner in response to the mask shape shown by FIG. 3A. As will be understood from the above description, by adjusting the level of the reference voltage Vr, it is possible to make correspondence in a relationship between the mask signal S3 and the mask shape appropriate.

Returning to FIG. 1, the mask signal S3 acquired as described above, and being an electrical signal corresponding to the actual mask shape of the electron beam exposure mask M, is input to one input of a signal comparator 6. In order to check whether or not the actual mask shape is formed as planned using the mask signal S3, that is, to check whether or not there are defects in the actual mask shape, a CAD signal S4 formed based on CAD data DT used to make the electron beam exposure mask M stored in the memory 7 is supplied from the CAD signal generator 8 to the other input of the signal comparator 6.

In order to synchronize the CAD signal S4 with the mask signal S3 from the CAD data DT stored in the memory 7, a coordinate signal S5 is input to the CAD signal generator 8 from the scanning signal generator 3. The coordinate signal S5 is formed inside the scanning signal generator 3 based on the scanning signal S1, and represents coordinates of scanning points of the electron beam 2B for scanning using the scanning signal S1 at that time. At the CAD signal generator 8, CAD data for coordinate positions represented by this coordinate signal S5 are read from the memory 7 and output as the CAD signal S4.

Referring to FIG. 3, FIG. 3D represents a CAD graphic, being the mask shape estimated based on the CAD data DT. Accordingly, the waveform of the CAD signal S4 is as shown in FIG. 3E. The mask signal S3 and the CAD signal S4 synchronized with the mask signal S4 are input to the signal comparator 6, and the levels of the signals are compared. If the levels of the two signals S3 and S4 match, the output of the signal comparator 6 is a low level, but if the levels of the two signals S3 and S4 do not match, the output is a high level. Accordingly, with the example shown in FIG. 3, as shown in FIG. 3A, the output of the signal comparator 6 is at a high level at portions where the two signals S3 and S4 do not match corresponding to missing portions MX that are missing from the actual mask shape.

Thus a defect signal S6 that is a high level only at portions where there are defects in the mask shape of the electron beam exposure mask is output from the signal comparator 6, and check result data according to the defect signal S6 are stored in the defect storage memory 9.

With this embodiment, the coordinate signal S5 is supplied to the defect storage memory 9, whether or not there is a defect is determined for coordinate positions on the electron beam exposure mask M sequentially represented by the coordinate signal S5 using information from the defect signal S6, and defect result data is stored as data of “0” or “1”.

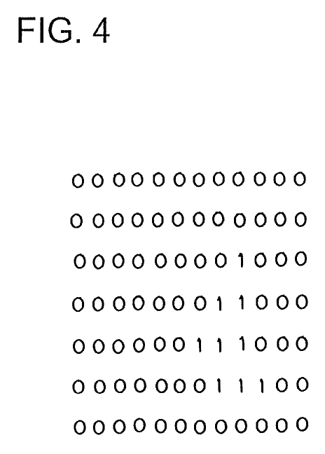

FIG. 4 shows one example of check result data acquired in this way. The check result data is allocated for all the coordinate points of the electron beam exposure mask M, and is “0” if there is no defect and “1” if there is a defect. Accordingly, by displaying this check result data on a display device, not shown, it becomes possible to immediately ascertain where defects have arisen on the electron beam exposure mask M.

Since the mask defect checking device 1 is constructed as described above, there is no need to acquire an optical image of the electron beam exposure mask M, and it is possible to promptly and accurately check whether or not there are defects in the electron beam exposure mask M using an electrical signal and a CAD signal based on transmission electrons acquired using electron beam scanning. Accordingly, in electron beam exposure mask checking for an electron beam exposure method aimed at manufacturing technology for patterns less than 0.1 μm, it is possible to realize high throughput, and it is possible to realize a reduction in the burden of checking cost in a mask checking process.

According to the present invention, as described above, there is no need to acquire an optical image of an electron beam exposure mask, and it is possible to promptly and accurately check whether or not there are defects in an electron beam exposure mask using an electrical signal and a CAD signal based on transmission electrons acquired using electron beam scanning. Accordingly, in electron beam exposure mask checking for an electron beam exposure method aimed at manufacturing technology for patterns less than 0.1 μm also, it is possible to realize high throughput, and it is possible to realize a reduction in the burden of checking cost in a mask checking process.

What is claimed is:

1. A mask defect checking device for electron beam exposure, for checking defects of a mask used in electron beam exposure, comprising:
an electron beam scanner for two dimensional scanning of a mask to be checked using an electron beam in response to a given scanning signal;
mask signal output means for outputting a mask signal in response to a mask shape based on transmission electrons passing through the mask from scanning of the electron beam;
CAD signal output means for outputting a CAD signal slowing a required mask shape in synchronism with output of the mask signal based CAD data for making the mask; and
comparison means for comparing the mask signal and the CAD signal, wherein
defects of the mask are checked based on output from the comparison means.

2. The mask defect checking device for electron beam exposure of claim 1, wherein the CAD signal output means synchronizes the mask signal and the CAD signal based on the scanning signal.

3. The mask defect checking device for electron beam exposure of claim 1, wherein the CAD data is stored in memory, and the CAD signal output means outputs the CAD signal by reading CAD data, for coordinate positions according to a coordinate signal representing coordinates for scanning points of the electron beam acquired based on the scanning signal, from the memory.

4. The mask defect checking device for electron beam exposure of claim 1, wherein the mask signal output means comprises a transmission electron detector for detecting the transmission electrons, and a sensitivity regulator for comparing an output signal from the transmission electron detector with a reference signal of a given fixed level to acquire the mask signal.

5. The mask defect checking device for electron beam exposure of claim 1, wherein mismatch information of the mask signal and the CAD signal from the comparison means is taken out as a defect signal.

6. The mask defect checking device for electron beam exposure of claim 5, wherein the defect signal is stored in memory.

7. A mask defect checking method for electron beam exposure, for checking defects of a mask used in electron beam exposure, comprising the steps of:
acquiring a mask signal corresponding to a mask shape based on the mask transmission electron signal acquired by two dimensional scanning of a mask to be checked using an electron beam, comparing the mask signal with a CAD signal corresponding to CAD graphics for making the mask, and checking for defects in the mask based on the comparison results.

8. The mask defect checking method for electron beam exposure of claim 7, wherein the mask signal and the CAD signal are synchronized based on a scanning signal for two-dimensional scanning of the electron beam.
